(19) United States
(12) Patent Application Publication
(10) Pub. No.: US 2016/0114104 A1
(43) Pub. Date: Apr. 28, 2016

(54) MEDICAMENT INFUSION SYSTEM AND METHODS OF USE THEREOF

(71) Applicant: Elwha LLC, Bellevue, WA (US)

(72) Inventors: Roderick A. Hyde, Redmond, WA (US); Jordin T. Kare, Seattle, WA (US); Elizabeth A. Sweeney, Seattle, WA (US); Clarence T. Tegreene, Mercer Island, WA (US)

(21) Appl. No.: 14/521,664

(22) Filed: Oct. 23, 2014

(51) Int. Cl. A61M 5/168

(52) U.S. Cl. CPC A61M 5/16804

(57) ABSTRACT

A medicament infusion system and methods of use thereof are disclosed herein for delivery of a medicament from a medicament source reservoir to a patient recipient. A medicament infusion system is disclosed that includes: a cylindrical tubing member having a medicament source connector configured to connect to a medicament source reservoir and a patient recipient connector configured to connect to a patient recipient; a medicament data transmitter operably connected to transmit medicament source data to the medicament source connector; a communication connection embedded within the wall of the cylindrical tubing member to transmit medicament source data from the medicament source connector to the patient recipient connector; a wireless tag operably connected to communicate with the patient recipient connector, the wireless tag configured to transmit the medicament source data; and a controller including circuitry to determine a connection status between the medicament source connector and the patient recipient connector.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

A medicament infusion system and methods of use thereof are disclosed herein for delivery of a medicament from a medicament source reservoir to a patient recipient. A medicament infusion system is disclosed that includes: a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; a medicament data transmitter operably connected to communicate with the medicament source connector, the medicament data transmitter configured to transmit medicament source data to the medicament source connector; a communication connection embedded within the wall of the cylindrical tubing member, the communication connection configured to transmit medicament source data from the medicament source connector to the patient recipient connector; a wireless tag operably connected to communicate with the patient recipient connector, the wireless tag configured to transmit the medicament source data; and a controller operably connected to receive medicament source data from the wireless tag, the controller including circuitry to determine a connection status between the medicament source connector and the patient recipient connector. In some aspects, the controller includes circuitry to receive patient recipient data to determine the connection status between the medicament source connector and the patient recipient connector. The patient recipient data may comprise at least one of a patient identifier, a patient treatment parameter, an identifier of a third party payer associated with the patient, and an identifier of a medical personnel associated with the patient. The patient treatment parameter may comprise at least one of an identifier of a source medicament prescribed for the patient, an indication that the source medicament is authorized for delivery to the patient by the medicament infusion system, a prescribed delivery time for the source medicament, a prescribed delivery dosage for the source medicament, a prescribed delivery rate for the source medicament, and a prescribed composition of the source medicament.

The system may include a signaling system operably connected to the controller and configured to transmit a wireless medicament delivery signal to the wireless tag, the medicament delivery signal comprising delivery instructional data for the source medicament based on the determined connection status between the medicament source connector and the patient recipient connector. The system may include one or more valves operably connected to the cylindrical tubing member, the one or more valves configured to modify flow of a medicament through the cylindrical tubing member between the medicament source reservoir and the patient recipient in response to the delivery instructional data. The communication connection may be configured to transmit the delivery instructional data from the patient recipient connector to the medicament source connector.

The medicament data transmitter may be configured to transmit the medicament source data to a first receiver on the medicament source connector. The communication connection may be configured to transmit the medicament source data from the medicament source connector to a second receiver on the patient recipient connector. The wireless tag may include, but is not limited to, RFID tag, an optical tag, or an ultrasonic tag. The system may include an alert signaling system in communication with the controller to notify a caregiver of the connection status between the medicament source connector and the patient recipient connector, wherein the controller receives and compares the patient recipient data and the medicament source data from the wireless tag to determine the connection status between the medicament source connector and the patient recipient connector. The medicament source data may include at least one of an identifier for the source medicament, a composition of the source medicament, a prescribed delivery rate of the source medicament, an actual delivery rate of the source medicament, an amount of the source medicament remaining in the medicament source reservoir, and a patient identifier for the prescribed source medicament. In some aspects of the system, a medicament is contained within the medicament source reservoir. The medicament data transmitter may be in contact with the medicament source reservoir. The medicament data transmitter may be communicatively linked to the medicament source reservoir. The wireless tag may include the patient recipient data, and is configured to transmit the patient recipient data to the controller. In some aspects of the system, the controller may be configured to receive the patient recipient data from an external database. The controller may be configured to receive the patient recipient data from an internal database. The controller may be included within a handheld electronic device. The controller may be included within an electronic device proximal to the patient recipient.

The wireless tag may be configured to transmit at least a portion of the medicament source data in response to reception of an RF signal by the wireless tag. In some aspects, the RF signal may comprise a request for a first portion of the medicament source data, and wherein the wireless tag is configured to transmit at least a second portion of the medicament source data corresponding to at least a part of the first portion of medicament source data. The medicament data transmitter may be configured to transmit the second portion of the medicament source data in response to reception of a wireless signal by the wireless tag. The medicament data transmitter may include an electrical transmitter or an optical transmitter configured to transmit the medicament source data electrically or optically, to the medicament source connector. The medicament data transmitter may include a wireless transmitter configured to transmit the medicament source data wirelessly by near field propagation to the medicament source connector. The wireless tag may be configured to receive the data electrically, optically, or wirelessly from the patient recipient connector. The system may include a signaling light operably connected to the controller to indicate a successful data linkage between the medicament data transmitter, the medicament source connector, the patient recipient connector, and the wireless tag. The wireless tag may be configured to be located on or near the patient recipient and adjacent to the patient recipient connector of the tubing member. The wireless tag may be on a cannula at least a portion of which is configured to be inserted within the patient recipient.

The system may include one or more intermediate cylindrical tubing members having a second communication connection embedded within a second wall defining a second interior passage, the second cylindrical tubing member having one or more intermediate connectors configured to connect the one or more intermediate cylindrical tubing members between the medicament source connector and the patient recipient connector, wherein the one or more intermediate connectors are configured to transmit the medicament source data between the intermediate cylindrical tubing members, the medicament source data configured to be transmitted between the medicament source connector and the patient recipient connector via the second communication connection and the one or more intermediate connectors. The medicament source data may be communicated to the wireless tag associated with the patient recipient. The system may include two or more intermediate cylindrical tubing members fluidically and communicatively coupled to the medicament source connector and the patient recipient connector on the cylindrical tubing member by two or more intermediate connectors. The two or more intermediate connectors may be configured to transmit the source medicament data through a second wall of the intermediate cylindrical tubing members. The communication connection may include electrical conductors embedded within the wall of the tubing member. The communication connection may include an optical waveguide embedded within the wall of the tubing member. The optical waveguide may be embedded in at least a portion of the wall of the tubing member. The optical waveguide may be embedded in at least a portion of the interior passage of the tubing member. The system may include an optical source to transmit the data through the optical waveguide.

A method is disclosed that includes: transmitting medicament source data along a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; transmitting the medicament source data from a medicament data transmitter to the medicament source connector, wherein the medicament data transmitter is operably connected to communicate with the medicament source connector; transmitting the medicament source data on a communication connection embedded within the wall of the cylindrical tubing member, the communication connection operably connected to communicate between the medicament source connector and the patient recipient connector; transmitting the medicament source data from the patient recipient connector operably connected to communicate to a wireless tag associated with the patient recipient; transmitting the medicament source data from the wireless tag operably connected to communicate to a controller; and determining at the controller a connection status between the medicament source connector and the patient recipient connector.

The method may include transmitting patient recipient data to the controller; and determining at the controller the connection status between the medicament source connector and the patient recipient connector. The method may include transmitting from a signaling system operably connected to the controller, a wireless medicament delivery signal transmitted to the wireless tag, the wireless medicament delivery signal comprising delivery instructional data for the source medicament based on the determined connection status between the medicament source connector and the patient recipient connector. The method may include receiving and comparing the patient recipient data and the medicament source data from the wireless tag at the controller to determine the connection status between the medicament source connector and the patient recipient connector; and notifying a caregiver of the connection status between the medicament source connector and the patient recipient connector by a signaling system. The method may include modifying flow of a medicament between the medicament source reservoir and the patient recipient with one or more valves in response to the delivery instructional data.

The method may include transmitting via the communication connection the delivery instructional data from the patient recipient connector to the medicament source connector. The patient recipient data may include at least one of a patient identifier, a patient treatment parameter, an identifier of a third party payer associated with the patient, and an identifier of a medical personnel associated with the patient. The patient treatment parameter may include at least one of an identifier of a source medicament prescribed for the patient, an indication that the source medicament is authorized for delivery to the patient by the medicament infusion system, a prescribed delivery time for the source medicament, a prescribed delivery dosage for the source medicament, a prescribed delivery rate for the source medicament, and a prescribed composition of the source medicament. The method may include providing one or more intermediate cylindrical tubing members having a second communication connection embedded within a second wall defining a second interior passage, the second cylindrical tubing member having one or more intermediate connectors configured to connect the one or more intermediate cylindrical tubing members between the medicament source connector and the patient recipient connector, wherein the one or more intermediate connectors are configured to transmit the medicament source data between the intermediate cylindrical tubing members, the medicament source data configured to be transmitted between the medicament source connector and the patient recipient connector via the second communication connection and the one or more intermediate connectors. The method may include communicating the medicament source data to the wireless tag associated with the patient recipient. The method may include fluidically and communicatively coupling through two or more walls of two or more intermediate cylindrical tubing members by two or more intermediate connectors to the medicament source connector and the patient recipient connector on the cylindrical tubing member.

The method may include communicatively linking the source reservoir to the medicament data transmitter wherein the source reservoir comprises the medicament data transmitter. The method may include transmitting via the medicament data transmitter at least a portion of the medicament source data in response to reception of a wireless signal from the wireless tag. The method may include transmitting the data through the medicament data transmitter electrically or optically to the medicament source connector. The method may include transmitting the data through the medicament data transmitter wirelessly by near field propagation to the medicament source connector. The method may include receiving data electrically, optically, or wirelessly by the RFID tag at the patient recipient connector. The method may include signaling via a signaling light a successful data linkage between the medicament data transmitter, the medicament source connector, the patient recipient connector, and the wireless tag. The method may include locating the wireless tag on or near a patient and adjacent to the patient recipient connector of the tubing member. The method may include locating the wireless tag on a cannula at least a portion of which is configured to be inserted within the patient recipient.

The method may include embedding electrical conductors on the communication connection within the wall of the tubing member. The method may include embedding an optical waveguide on the communication connection within the wall of the tubing member. The method may include transmitting light through the optical waveguide to transmit data through at least a portion of a wall of the cylindrical tubing member. The method may include transmitting light through the optical waveguide to transmit data through at least a portion of the interior passage of the cylindrical tubing member.

The method may include providing medicament within the medicament source reservoir. The method may include initiating flow of the medicament from the medicament source reservoir to the patient recipient.

A device is disclosed that includes: a system including a signal-bearing medium including, one or more instructions for transmitting medicament source data along a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; one or more instructions for transmitting the medicament source data from a medicament data transmitter to the medicament source connector, wherein the medicament data transmitter is operably connected to communicate with the medicament source connector; one or more instructions for transmitting the medicament source data on a communication connection embedded within the wall of the cylindrical tubing member, the communication connection operably connected to communicate between the medicament source connector and the patient recipient connector; one or more instructions for transmitting the medicament source data from the patient recipient connector operably connected to communicate to a wireless tag associated with the patient recipient; one or more instructions for transmitting the medicament source data from the wireless tag operably connected to communicate to a controller; and one or more instructions for determining a connection status between the medicament source connector and the patient recipient connector at the controller.

A medicament infusion system is disclosed that includes: a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; a medicament data transmitter operably connected to communicate with the medicament source connector, the medicament data transmitter configured to transmit medicament source data to the medicament source connector; an optical waveguide embedded within the wall of the cylindrical tubing member, the optical waveguide configured to transmit medicament source data from the medicament source connector to the patient recipient connector; a wireless tag operably connected to communicate with the patient recipient connector, the wireless tag configured to transmit the medicament source data; a controller operably connected to receive medicament source data from the wireless tag, the controller including circuitry to determine a connection status between the medicament source connector and the patient recipient connector.

A medicament infusion system is disclosed that includes: a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; a medicament data transmitter operably connected to communicate with the medicament source connector, the medicament data transmitter configured to transmit medicament source data to the medicament source connector; an electrical connection embedded within the wall of the cylindrical tubing member, the electrical connection configured to transmit medicament source data from the medicament source connector to the patient recipient connector; a wireless tag operably connected to communicate with the patient recipient connector, the wireless tag configured to transmit the medicament source data; and a controller operably connected to receive medicament source data from the wireless tag, the controller including circuitry to determine a connection status between the medicament source connector and the patient recipient connector.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A and 1B depict a diagrammatic view of an aspect of a medicament infusion system.

FIG. 2 depicts a diagrammatic view of an aspect of a medicament infusion system.

FIG. 3 depicts a diagrammatic view of an aspect of a medicament infusion system.

FIGS. 4A and 4B depict a diagrammatic view of an aspect of a medicament infusion system.

FIG. 5 depicts a method that includes providing a medicament infusion system.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

A medicament infusion system and methods of use thereof are disclosed herein for delivery of a medicament from a medicament source reservoir to a patient recipient. A medicament infusion system is disclosed that includes: a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; a medicament data transmitter operably connected to communicate with the medicament source connector, the medicament data transmitter configured to transmit medicament source data to the medicament source connector; a communication connection embedded within the wall of the cylindrical tubing member, the communication connection configured to transmit medicament source data from the medicament source connector to the patient recipient connector; and a wireless tag operably connected to communicate with the patient recipient connector, the wireless tag configured to transmit the medicament source data; a controller operably connected to receive medicament source data from the wireless tag, the controller including circuitry to determine a connection status between the medicament source connector and the patient recipient connector. The controller may include circuitry to receive patient recipient data to determine the connection status between the medicament source connector and the patient recipient connector. In some aspects, the wireless tag includes, but is not limited to, an RFID tag, an optical tag, or an ultrasonic tag.

The medicament infusion system including the communication connection may include embedded conductors in the communication connection embedded within the wall of the tubing member to transmit the data between the medicament source connector and the patient recipient connector. The medicament infusion system including the communication connection may include an optical waveguide embedded within the wall of the tubing member to transmit the data between the medicament source connector and the patient recipient connector. This data may be the data regarding the source medicament or may be the RF signal (query) received by the wireless tag with regard to medicament source data and patient recipient data.

The medicament infusion system may include a signaling system operably connected to the controller and configured to transmit an RF medicament delivery signal to the wireless tag. The medicament delivery signal may comprise delivery instructional data for the source medicament based on the determined connection status between the medicament source connector and the patient recipient connector. The medicament delivery signal based on the determined connection status may control opening or closing a flow valve within the medicament infusion system, wherein the controllable flow valve is located between the medicament source reservoir and a cannula inserted into the patient.

A method is disclosed that includes: transmitting medicament source data along a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; transmitting the medicament source data from a medicament data transmitter to the medicament source connector, wherein the medicament data transmitter is operably connected to communicate with the medicament source connector; transmitting the medicament source data on a communication connection embedded within the wall of the cylindrical tubing member, the communication connection operably connected to communicate between the medicament source connector and the patient recipient connector; transmitting the medicament source data from the patient recipient connector operably connected to communicate to a wireless tag associated with the patient recipient; transmitting the medicament source data from the wireless tag operably connected to communicate to a controller; and determining a connection status between the medicament source connector and the patient recipient connector at the controller.

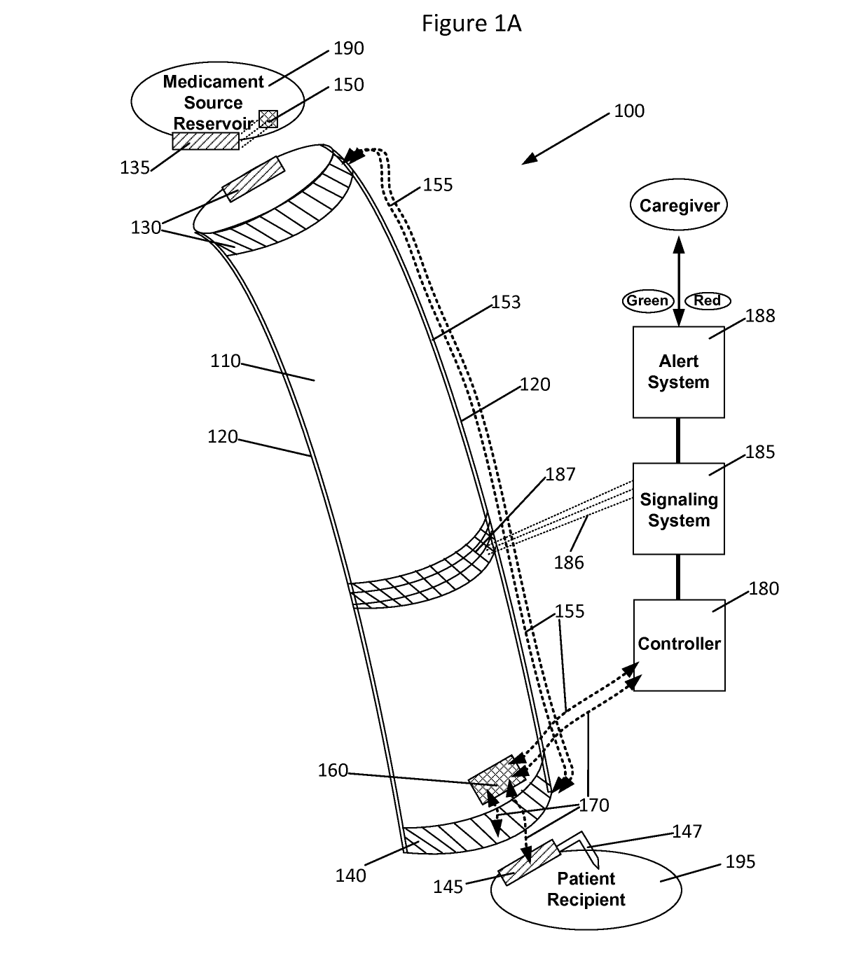

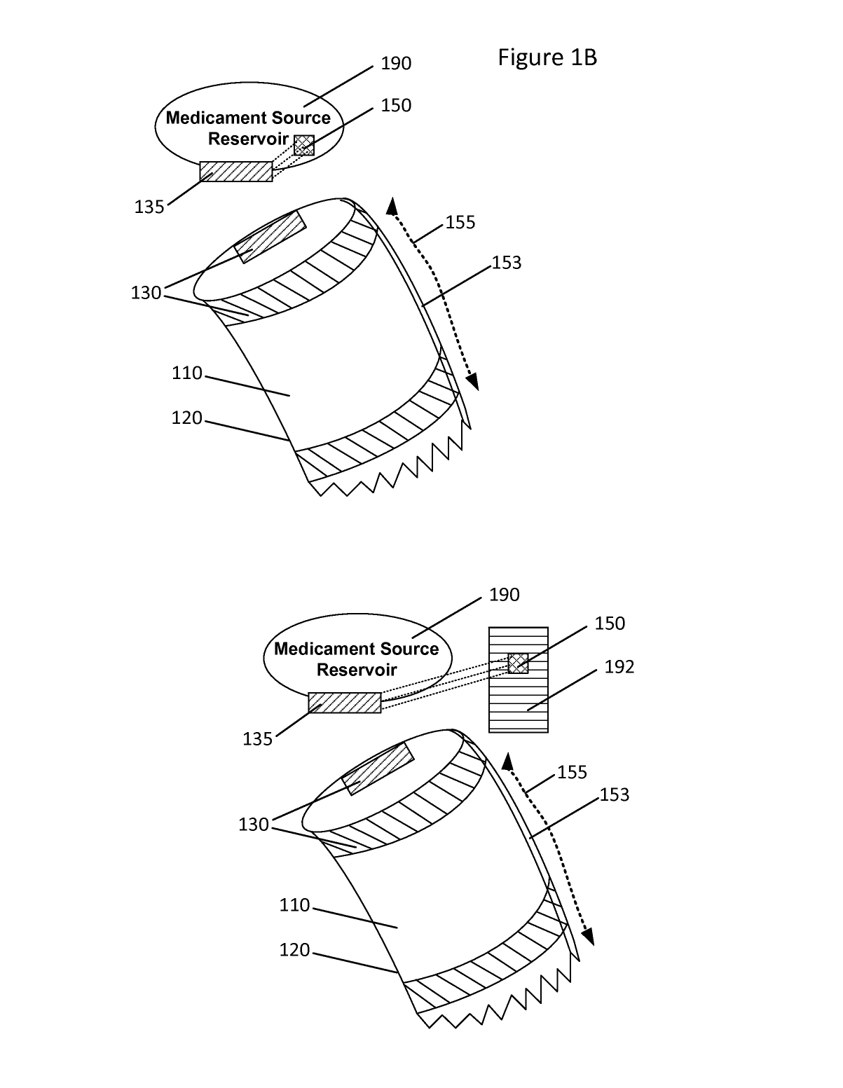

FIG. 1A depicts a diagrammatic view of an aspect of a medicament infusion system 100 to connect a medicament source reservoir 190 to a patient recipient 195. The medicament infusion system 100 includes a cylindrical tubing member 110 having a wall 120 defining an interior passage, a medicament source end, and a patient recipient end, the tubing member 110 having a medicament source connector 130 configured to connect a connector 135 on a medicament source reservoir 190 at the medicament source end and a patient recipient connector 140 configured to connect to connector 145 on a device, e.g., catheter 147, on a patient recipient 195 at the patient recipient end; a medicament data transmitter 150 operably connected to communicate with the medicament source connector 130 and the connector 135 on the medicament source reservoir, the medicament data transmitter 150 configured to transmit medicament source data 155 to the medicament source connector 130; a communication connection 153 embedded within the wall 120 of the cylindrical tubing member 110, the communication connection 153 configured to transmit medicament source data 155 from the medicament source connector 130 to the patient recipient connector 140; a wireless tag 160, e.g., an RFID tag, operably connected to communicate with the patient recipient connector 140 and the connector 145 on device 147, the wireless tag 160 configured to transmit the medicament source data 155; and a controller 180 operably connected to receive medicament source data 155 from the wireless tag 160, the controller 180 including circuitry to determine a connection status between the medicament source reservoir 190 and the patient recipient 195. In some aspects, the controller 180 is configured to receive patient recipient data 170 to determine the connection status between the medicament source reservoir 190 and the patient recipient 195. In some aspects, the controller 180 is an RFID reader. In some aspects, the controller/RFID reader may be a handheld device or may be located in the vicinity of the wireless tag. The medicament source connector 130 at the medicament source end of the tubing may have interacting components 130 and 135 at the medicament source reservoir 190. The patient recipient connector 140 at the patient recipient end of the tubing may have interacting components 140 and 145 at the patient recipient 195, e.g., at a catheter 147 to be inserted into the patient. In some aspects, the patient recipient connector 140 is attached to a catheter 145 configured to be inserted into the patient recipient 195. In some aspects, the wireless tag 160 may include, but is not limited to, an RFID tag, an optical tag, or an ultrasonic tag.

The medicament infusion system 100 includes a signaling system 185 operably connected to the controller 180 and configured to transmit an RF medicament delivery signal to the wireless tag 160, the medicament delivery signal comprising delivery instructional data for the source medicament based on the determined connection status between the medicament source reservoir 190 and the patient recipient 195.

The communication connection 153 embedded within the wall 120 is configured to transmit medicament source data 155 from the medicament source connector 130 to the patient recipient connector 140; The communication connection 153 is further configured to transmit the delivery instructional data 155 from the patient recipient connector 140 to the medicament source connector 130. The medicament delivery signal 186 based on the determined connection status may signal controller 180 and signaling system 185 to control opening or closing a flow valve 187 within the medicament infusion system, wherein the controllable flow valve 187 is located between the medicament source reservoir and a cannula inserted into the patient.

The signaling system 185 in communication with the controller 180 receives and compares the patient recipient data 170 and the medicament source data 155 from the wireless tag 160 to determine the connection status between the medicament source reservoir 190 and the patient recipient 195. The determined connection status indicates whether the correct medicament is being delivered to the correct patient recipient. Based on the determined connection status, the medicament delivery signal 186 transmits delivery instructional data for the source medicament in the medicament source reservoir based on the determined connection status between the medicament source connector 130 and the patient recipient connector 140. If the connection status determines that the patient recipient data 170 and the medicament source data 155 agree, the medicament delivery signal 186 transmits delivery instructional data to open controllable flow valves 187 at the medicament reservoir and/or at the patient recipient connector for delivery of the source medicament at the prescribed dosage and prescribed rate. The signaling system 185 in communication with the controller 180 will notify a caregiver by an alert system 188 of a connection status between the medicament source reservoir 190 and the patient recipient 195. The caregiver will be alerted by the alert system 188 to the correct connection status (green) or incorrect connection status (red) prior to initiating medicament infusion. The caregiver may also check on the status of medicament infusion, e.g., flow rate and total delivery volume, during the infusion process through the controller 180 and alert system 188.

In some aspects, the medicament infusion system 100 includes a signaling light from an alert signaling system 188 indicating a successful data linkage (green) or unsuccessful data linkage (red) between the medicament data transmitter 150, the medicament source connector 130, the patient recipient connector 140, and the wireless tag 160. In some aspects of the medicament infusion system 100, the wireless tag 160 is configured to be located on or near the patient recipient 195 and adjacent to the patient recipient connector 140 of the tubing member 110. In some aspects, the wireless tag 160 is on a cannula 165 configured to be inserted within the patient recipient 195. In some aspects the wireless tag 160 is located on or near the tubing member 110.

FIG. 1B depicts a diagrammatic view of an aspect of a medicament infusion system 100. In some aspects, the medicament data transmitter 150 is in contact with the medicament source reservoir 190. In some aspects, the medicament data transmitter 150 is communicatively linked to the medicament source reservoir 190 from a remote location 192.

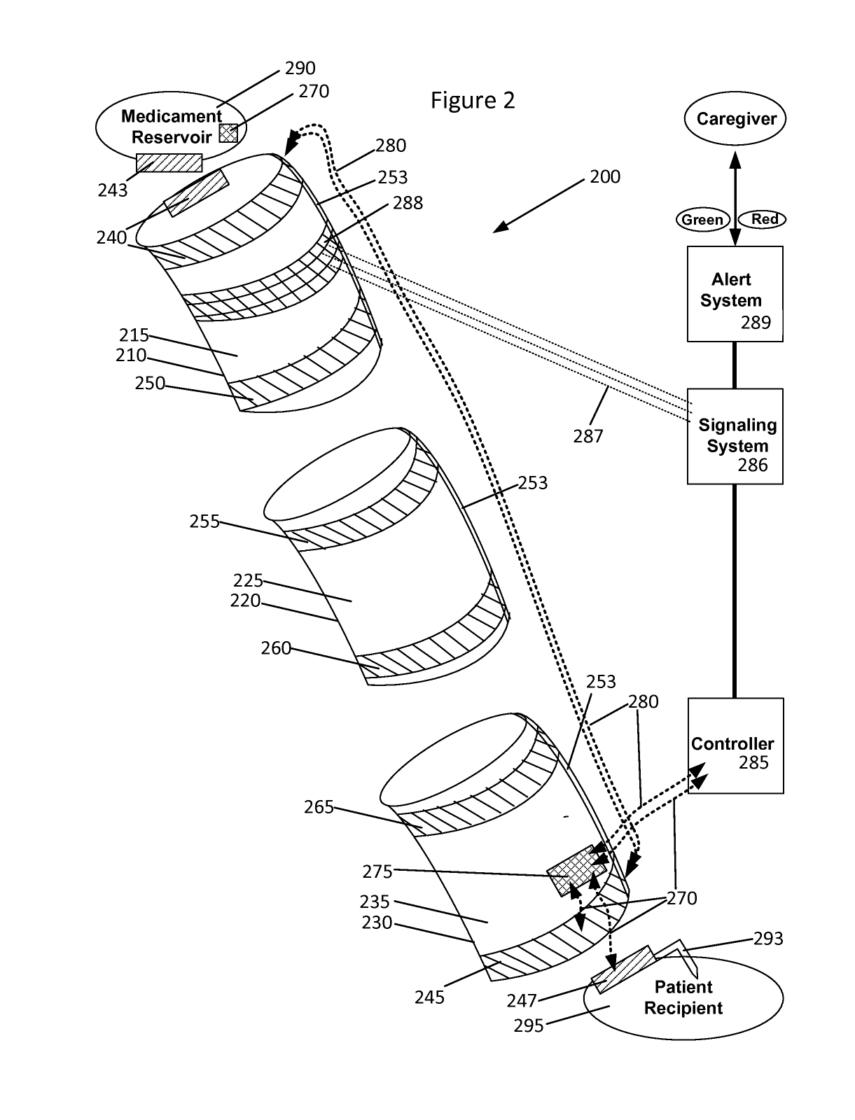

FIG. 2 depicts a diagrammatic view of an aspect of a medicament infusion system 200. The medicament infusion system 200 includes a cylindrical tubing member 215, 225, 235 having a wall 210, 220, 230 defining an interior passage, a medicament source end, and a patient recipient end, the tubing member 215, 225, 235 having a medicament source connector 240 configured to connect a connector 243 on a medicament source reservoir 290 at the medicament source end and a patient recipient connector 245 configured to connect to connector 247 on a device, e.g., catheter 293, on a patient recipient 295 at the patient recipient end. In some aspects, the medicament infusion system 200 includes two or more intermediate connectors 250, 255, 260, 265 configured to connect one or more intermediate cylindrical tubing members 215, 225, 235 between the first connector 240 at the medicament source end and the patient recipient connector 245, wherein the one or more intermediate connectors 250, 255, 260, 265 are configured to transmit the medicament source data 280 from the medicament source connector 240 at the medicament source end 290, transmitted between adjacent intermediate cylindrical tubing members within the walls 210, 220, 230 of intermediate cylindrical tubing members, and transmitted to the patient recipient connector 245 at the patient recipient end 295. The patient recipient connector may contact the patient at a venous catheter 293.

A communication connection 253 may be embedded within the walls 210, 220, 230 of the cylindrical tubing members 215, 225, 235, the communication connection 253 configured to transmit medicament source data 280 from the medicament source connector 240 to the patient recipient connector 245. A wireless tag 275, e.g., an RFID tag, may be operably connected to communicate with the patient recipient connector 245, the wireless tag 275 configured to transmit the medicament source data 280; and a controller 285 may be operably connected to receive medicament source data 280 from the wireless tag 275, the controller 285 including circuitry to determine a connection status between the medicament source reservoir 290 and the patient recipient 295. In some aspects, the controller 285 is configured to receive patient recipient data 270 to determine the connection status between the medicament source reservoir 290 and the patient recipient 295. In some aspects, the controller 285 is an RFID reader. In some aspects, the controller/RFID reader 285 may be a handheld device or may be located in the vicinity of the wireless tag 275. The medicament source connector 240 at the medicament source end of the tubing may have interacting components 240 at the medicament source reservoir 290. The patient recipient connector 245 at the patient recipient end of the tubing may have interacting components 245 at the patient recipient 295, e.g., at a catheter 293 to be inserted into the patient.

The medicament infusion system 200 includes a signaling system 286 operably connected to the controller 285 and configured to transmit an RF medicament delivery signal to the wireless tag 275, the medicament delivery signal comprising delivery instructional data for the source medicament based on the determined connection status between the medicament source reservoir 290 and the patient recipient 295. The communication connection 253 is configured to transmit the delivery instructional data 280 from the patient recipient connector 245 to the medicament source connector 240. The medicament delivery signal 287 based on the determined connection status may control opening or closing a flow valve 288 within the medicament infusion system, wherein the controllable flow valve 288 is located between the medicament source reservoir 290 and a cannula 293 inserted into the patient. In some aspects, the medicament infusion system 200 includes a signaling light from an alert signaling system 289 indicating a successful data linkage between the medicament data transmitter 270, the medicament source connector 240, the patient recipient connector 245, and the wireless tag 275.

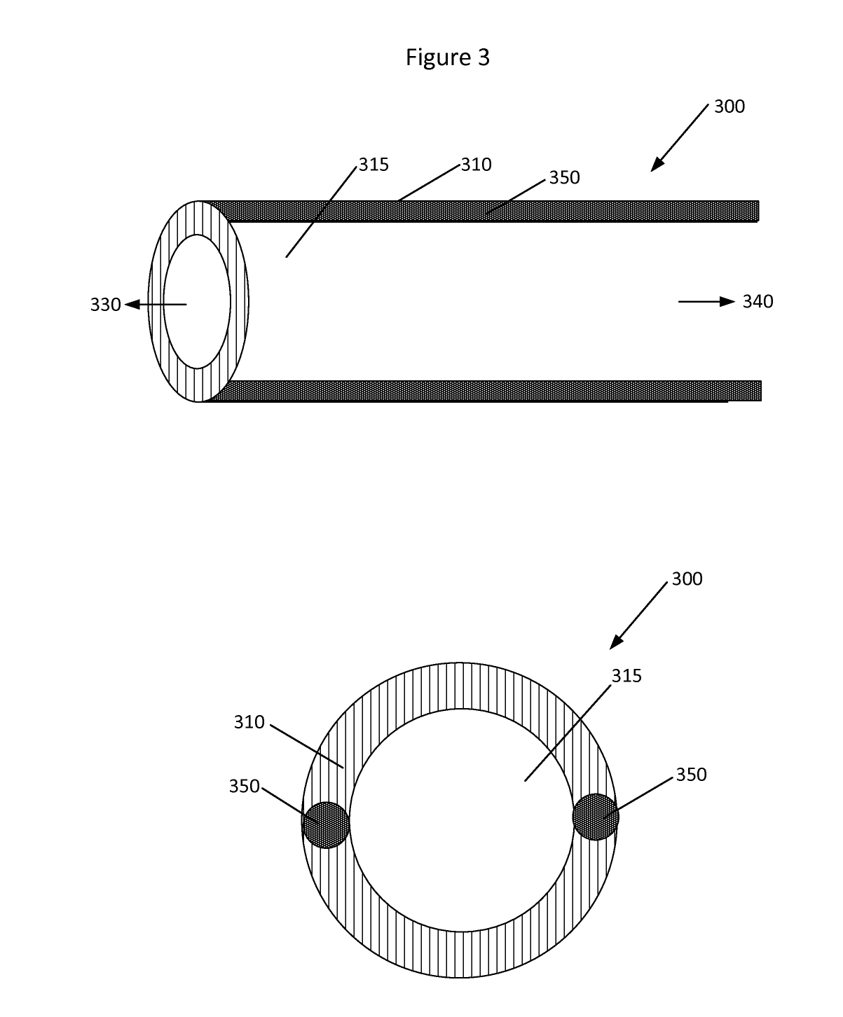

FIG. 3 depicts a diagrammatic cross sectional view of an aspect of a medicament infusion system 300 including a section of the tubing member 315. In some aspects, the medicament infusion system 300 includes embedded conductors 350 in the wall 310 of the tubing member 315 to transmit the data between the medicament source connector (toward directional arrow 330) and the patient recipient connector (toward directional arrow 340).

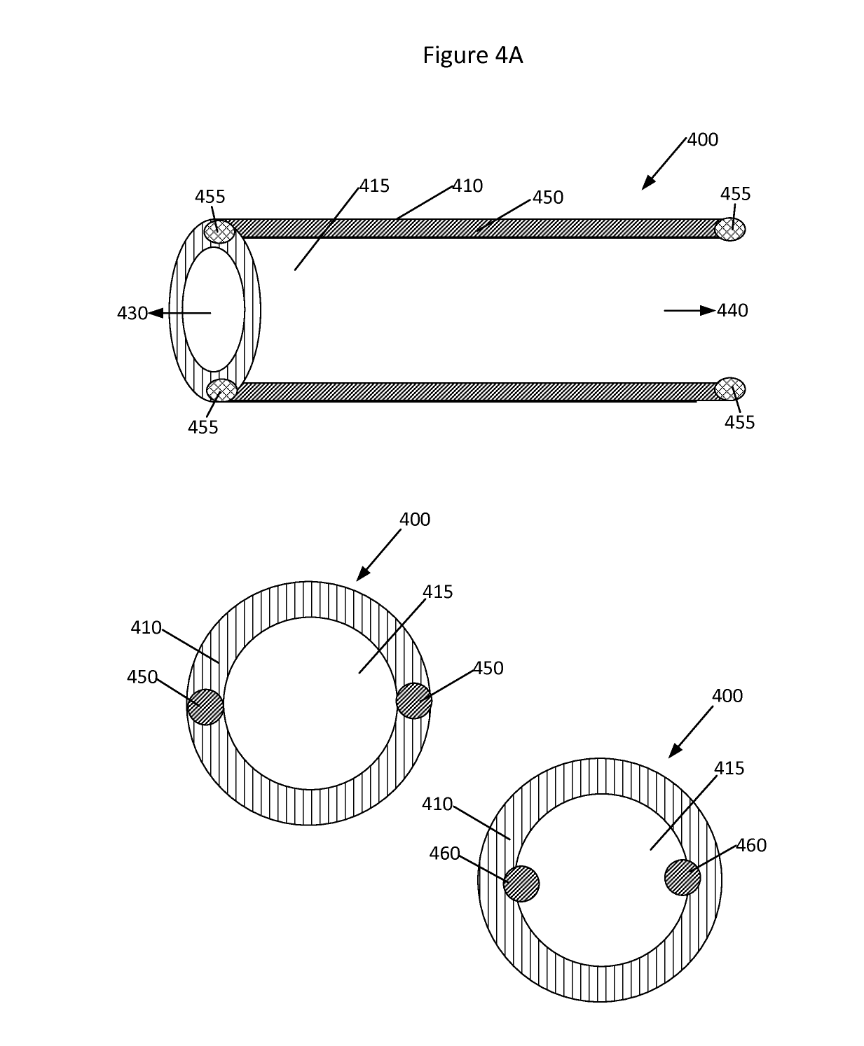

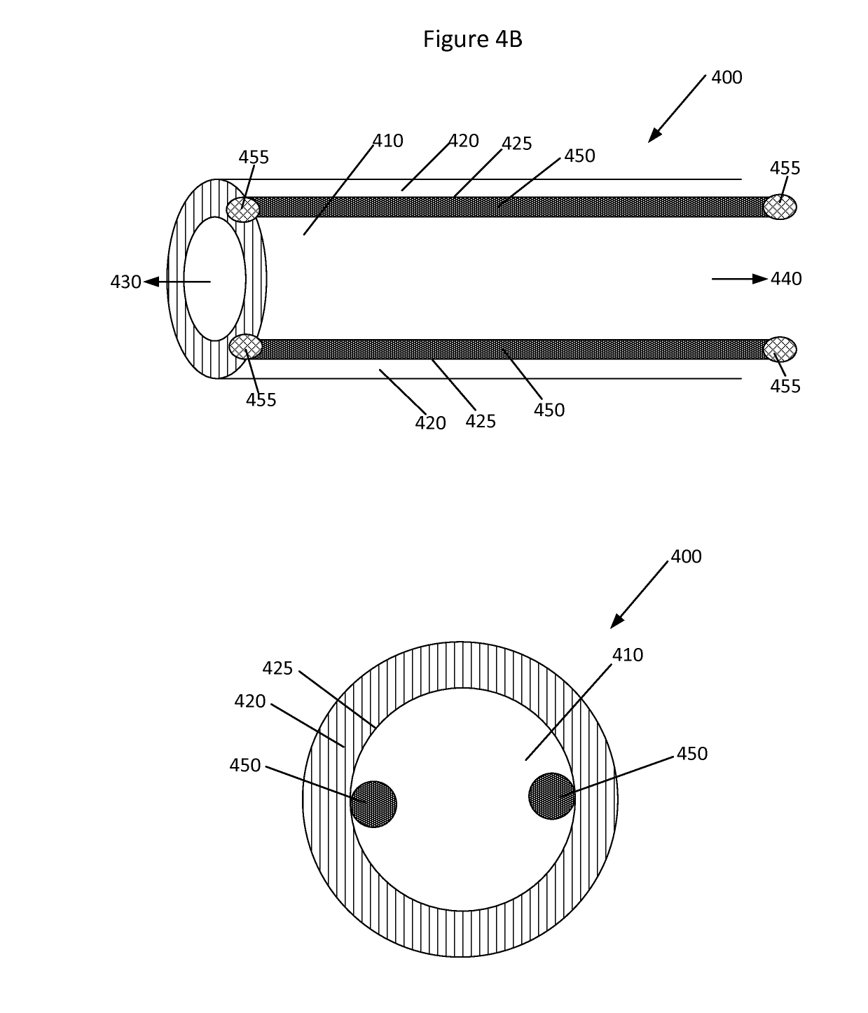

FIG. 4A depicts a diagrammatic cross sectional view of an aspect of a medicament infusion system 400 including a section of the tubing member 415. In some aspects, the medicament infusion system 400 includes an optical waveguide 450, 460 embedded in the wall 420 of the tubing member 410 to transmit the data between the medicament source connector 430 and the patient recipient connector 440. The optical waveguide 450, 460 is embedded within at least a portion of the wall 420 of the tubing member 410. The optical waveguide 450 may be completely embedded 450 within the wall 420, or the optical waveguide 460 may be partially embedded 460 within the wall 410. The medicament infusion system 400 includes an optical source 455 to transmit the data through the optical waveguide 450, 460. The medicament infusion system 400 includes an optical source 455 to transmit the data through the optical waveguide 450, 460. The optical source 455 may be on one or both ends of the optical waveguide 450, 460 to transmit the data bidirectionally from the medicament source connector 430 to the patient recipient connector 440 and from patient recipient connector 440 to the medicament source connector 430.

FIG. 4B depicts a diagrammatic cross sectional view of an aspect of a medicament infusion system 400 including a section of the tubing member 415. In some aspects, the medicament infusion system 400 includes an optical waveguide 450, wherein the optical waveguide 450 is within an interior passage inside the cylindrical tubing member 410 wherein the tubing member 410 has a wall 420 defining an interior passage. The optical waveguide 450 contacts at least a portion of the interior passage wall 425 of the tubing member 410 to transmit the data through the optical waveguide 450 between the medicament source connector 430 and the patient recipient connector 440. The medicament infusion system 400 includes an optical source 455 to transmit the data bi-directionally or uni-directionally through the optical waveguide 450.

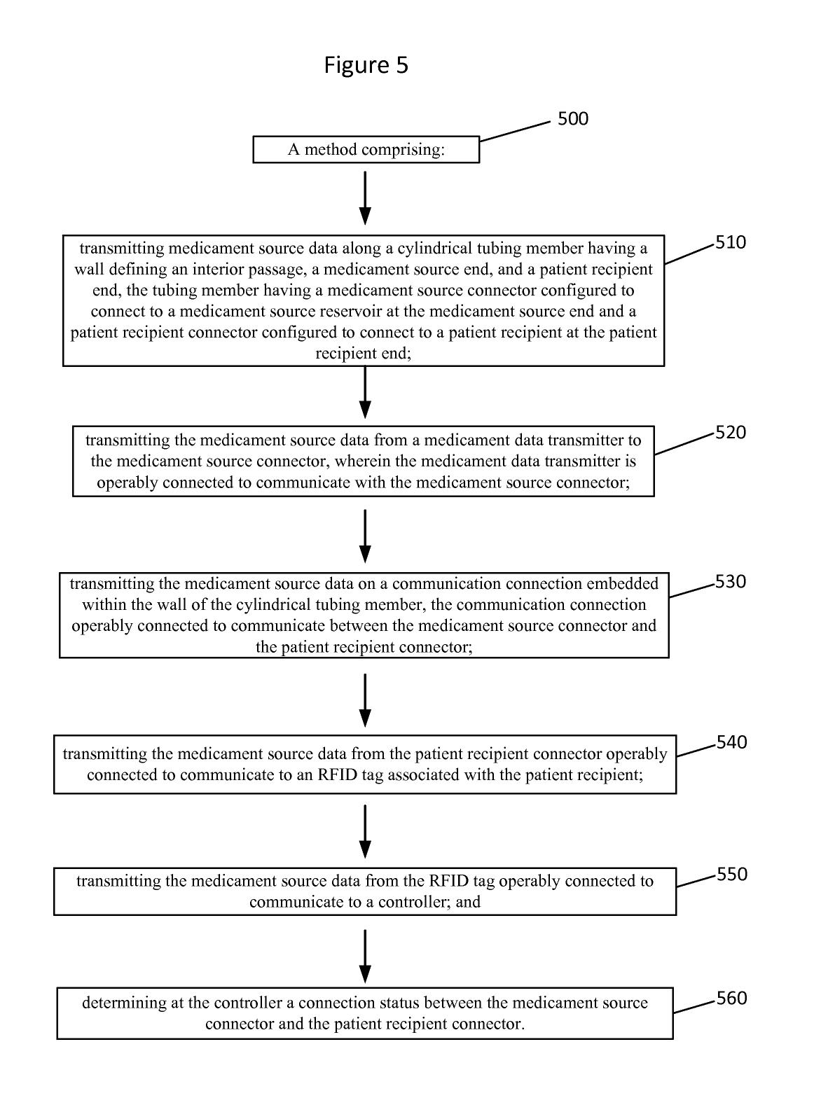

FIG. 5 depicts a diagrammatic view of a method 500 of using a medicament infusion system. In some aspects, the method 500 includes: transmitting 510 medicament source data along a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end; transmitting 520 the medicament source data from a medicament data transmitter to the medicament source connector, wherein the medicament data transmitter is operably connected to communicate with the medicament source connector; transmitting 530 the medicament source data on a communication connection embedded within the wall of the cylindrical tubing member, the communication connection operably connected to communicate between the medicament source connector and the patient recipient connector; transmitting 540 the medicament source data from the patient recipient connector operably connected to communicate to a wireless tag associated with the patient recipient; transmitting 550 the medicament source data from the wireless tag operably connected to communicate to a controller; and determining 560 at the controller a connection status between the medicament source connector and the patient recipient connector.

### Fiber-Optic Communication Through a Cylindrical Tubing Wall of a Medicament Infusion System

The medicament infusion system may include a fiber-optic communication system comprising an optical transmitter to convert an electrical signal into an optical signal. The optical signal is sent through an optical fiber, a cable containing bundles of multiple optical fibers that is routed through conduits embedded within a cylindrical tubing wall of a medicament infusion system. Multiple types of amplifiers and an optical receiver at the patient recipient end of the cylindrical tubing wall are utilized to recover the signal as an electrical signal. The information transmitted is typically digital information generated regarding data to match patient identity with prescribed medication for that patient as well as dosage, rate of flow and the status of drug infusion.

Optical Transmitters

The optical transmitters may include, for example, light-emitting diodes (LEDs) and laser diodes. LEDs produce incoherent light, while laser diodes produce coherent light. For use in optical communications, semiconductor optical transmitters are designed to be compact, efficient, and reliable, while operating in an optimal wavelength range, and directly modulated at high frequencies.

Communications LEDs may be made from a variety of laser-emitting materials including, but not limited to, Indium gallium arsenide phosphide (InGaAsP) or gallium arsenide (GaAs). VCSEL (Vertical Cavity Surface Emitting Laser) devices offer improved speed, power and spectral properties. Common VCSEL devices couple to multi-mode fiber. Classes of semiconductor laser transmitters used in fiber optics include VCSEL (Vertical-Cavity Surface-Emitting Laser), Fabry-Pérot and DFB (Distributed Feed Back).

Optical Receivers

An optical receiver includes a photodetector, which converts light into electricity using the photoelectric effect. The primary photodetectors for telecommunications are made from Indium gallium arsenide. The photodetector may be a semiconductor-based photodiode. Several types of photodiodes include p-n photodiodes, p-i-n photodiodes, and avalanche photodiodes. Metal-semiconductor-metal (MSM) photodetectors are also used due to their suitability for circuit integration in regenerators and wavelength-division multiplexers.

Optical-electrical converters may be coupled with a transimpedance amplifier and a limiting amplifier to produce a digital signal in the electrical domain from the incoming optical signal, which may be attenuated and distorted while passing through the channel. Further signal processing such as clock recovery from data (CDR) performed by a phase-locked loop may also be applied before the data is passed on. http://en.wikipedia.org/wiki/Fiber-optic_communication#Receivers

### Microprocessor, Microcircuitry, and Power Source

The medicament source connector of the medicament infusion system includes microcircuitry and a thin-film microbattery to empower the optical transmitter and transmit the medication source data. A microprocessor may be used in conjunction with a source of memory such as programmable read only memory (PROM), a timer, a demultiplexer, and a power source such as a microbattery. Status of the medicament reservoir or release pattern from the medicament reservoir is written directly into the PROM by the healthcare user. The PROM sends these instructions to the microprocessor. When the time for release has been reached as indicated by the timer, the microprocessor sends a signal corresponding to the address (location) of the medicament reservoir to the demultiplexer. The demultiplexer sends an input, such as an electric potential or current, to the medicament reservoir addressed by the microprocessor. A microbattery provides the power to operate the PROM, timer, and microprocessor, and provides the electric potential or current input that is directed to the medicament reservoir by the demultiplexer. The manufacture, size, and location of each of these components is dependent upon the requirements of a particular application. In one embodiment, the memory, timer, microprocessor, and demultiplexer circuitry are integrated directly onto the surface of the chip. The microbattery is attached to the other side of the chip and is connected to the device circuitry by vias or thin wires. However, in alternative aspects, one may use separate, prefabricated, component chips for memory, timing, processing, and demultiplexing. These components can be integrated with the medicament delivery microchip in a package such as a multi-chip module (MCM) or hybrid package, or they can be attached to the backside of the miniaturized delivery device with the battery. The size and type of prefabricated chips used depends on the overall dimensions of the medicament infusion system and the medicament reservoir. Second, activation of the medicament reservoir by the application of an electric current to the resistors can be controlled externally by remote controller, e.g., a handheld device including a controller used by healthcare workers. Much of the circuitry used for remote control is the same as that used in the preprogrammed method. The main difference is that the PROM is replaced by a signal receiver. A signal such as radio waves, microwaves, low power laser, or ultrasound is sent to the receiver by an external source, for example, computers or ultrasound generators. The signal is sent to the microprocessor where it is translated into an address for the medicament reservoir. Power is then directed through the demultiplexer to the medicament reservoir having the appropriate address. Third, a biosensor may be integrated into the microchip to detect molecules in the surrounding fluids. When the concentration of the molecules reaches a certain level, the sensor sends a signal to the microprocessor to query the medicament reservoir. The microprocessor directs power through the demultiplexer to the medicament reservoir. See e.g., U.S. Pat. No. 6,669,683 issued to Santini, Jr. et al. on Dec. 30, 2003 which is incorporated herein by reference.

### Medicament Infusion System Including Cylindrical Tubing Member Having an Embedded Conductor

The medicament infusion system may include tubing manufactured with embedded conductors, e.g., optical fibers, embedded within the wall of the infusion tubing or immediately adjacent to the wall of the infusion tubing. The medicament infusion tubing may include a thin flexible wall section extending between adjacent wraps of the bead. This thin flexible wall portion provides for flexibility for the tubing, while the bead provides support for the wall so that the tubing is kink-resistant and collapse-resistant. The conductors disposed within the bead are uniformly spaced apart as they extend helically along the tubing within the plateau portion captured within the bead. The conductors may be utilized to convey electrical signals transmitted along the helical configuration. Telemetry or instrumentation signals, e.g., light, fiber optic, or electrical, may be transmitted along the conductors. The spacing apart of these conductors ensures both insulation of the conductors from one another, a capacitance value between the conductors, uniformity of the capacitance value, and reduced mutual inductive coupling among the conductors. A twisted pair conductor set provides signal transmission along the infusion tubing. The medicament infusion tubing provides a dual-level disposition of the conductors at the bead of the tubing and a resulting greater number of conductors. See e.g., U.S. Patent Application No. 2014/0037276 by Carlson published on Feb. 6, 2014 which is incorporated herein by reference.

The medicament infusion system is connected to a medication source, for example, a medication fluid in an infusion bag with an outlet at the bottom with fittings for connection of infusion tubing, bubble traps and flow regulators. See e.g., U.S. Pat. No. 8,075,514 issued to Butterfield et al. on Dec. 13, 2011 and U.S. Patent Appl. No. 2014/0107579 by Lanigan et al. published on Apr. 17, 2014 which is incorporated herein by reference.

An infusion pump includes a mechanism for fluid administration by operative engagement with the pump. The fluid administration includes medicament infusion tubing, extending from the respective fluid container to the patient 48, upon which the pump acts to move fluid downstream to a catheter inserted into the patient. Specifically, a pumping mechanism acts as the flow control device of the pump to move fluid though the medicament infusion tubing. The pumping mechanism may be a “four finger” type pumping mechanism. The pumping mechanism may be a peristaltic pump that includes an upstream occluding finger, a primary pumping finger, a downstream occluding finger, and a secondary pumping finger. The “four finger” pumping mechanism and mechanisms used in other linear peristaltic pumps operate by sequentially pressing on a segment of the fluid conduit by means of the cam-following pumping fingers and valve fingers. The pressure is applied in sequential locations of the conduit, beginning at the upstream end of the pumping mechanism and working toward the downstream end. At least one finger is always pressing hard enough to occlude the conduit. One finger does not retract from occluding the tubing until the next one in sequence has already occluded the tubing; thus at no time is there a direct fluid path from the fluid container to the patient.

The infusion pump may also include two data reader devices, upstream data reader device, and downstream data reader device, for receiving information relating to the infusions, such as drug identification, patient identification, and optionally other information such as nurse identification. The data reader devices may comprise RFID readers (or receivers), optical readers (IR, visible, or UV), ultrasonic readers, or other wireless devices that are compatible with the data transmitting devices associated with the fluid containers and the data transmitting device associated with the patient. Although referred to as data reader devices or RFID readers or receivers, the data reader devices may also transmit interrogation signals to the RFID tag or transponder, optical tag or transponder (IR, visible, or UV), or ultrasonic tag or transponder associated with the medicament source container or with the patient recipient and as described below may also transmit data through a wall of the tubing member between a connector at a medicament source end and a connector at a patient recipient end, in either direction. Likewise, although referred to as data transmitting devices or RFID tags or RFID transponders, data transmitting devices may also receive or read data and may also re writable. In some aspects, the readers are operatively connected to the infusion pump and may be further provided as an integral part of the pump. While the readers may be located at a different location in the pump or may even be a separate component in operative connection with the pump, the upstream data reader device is generally located upstream from the pumping mechanism, that is, at a location between the medicament source container and the pumping mechanism. Likewise, the downstream data reader device is generally located at a downstream location with respect to the pumping mechanism, e.g., between the pumping mechanism and the patient recipient. The pump may include a single data device, either the upstream data reader device or the downstream data reader device.

The identification tag on the medicament source connector and/or on the patient recipient connector may be an RFID tag. The RFID tag may include an antenna and chip. The medicament source connector and/or on the patient recipient connector may be manufactured with a conductive ring. A chip may be electrically connected to the conductive ring. The RFID tag may be on the label medicament source or medicament source connector which may include additional information. For example, the information transmitted is typically digital information generated regarding data to match patient identity with prescribed medication for that patient as well as dosage, rate of flow and the status of drug infusion. See e.g., U.S. Pat. No. 8,075,514 issued to Butterfield et al. on Dec. 13, 2011 and U.S. Patent Appl. No. 2014/0107579 by Lanigan et al. published on Apr. 17, 2014 which is incorporated herein by reference.

Microchip Communication with an RFID Tag

The medicament infusion system includes a microchip is incorporated in the medication data transmitter from the medicament source and/or from medicament source connector that encodes medication information. A microchip is also incorporated in the patient recipient connector that encodes patient information, for example, the prescription information, patient identity, dose or concentration, schedule of administration, date and time. Microchip data carriers may be used to transmit identification information and information about drug sources.

The microchip may communicate with an RFID tag, optical (IR, visible, or UV) tag, or ultrasonic tag that includes a miniature integrated circuit and an antenna. A conductive loop may be implemented with a conductive component, e.g., conductive ink or fine breakable conducting wire such as copper. The conductive loop is deposited on the backing and is in electrical connection to microchip including the integrated circuit via conductive traces and pads. The conductive pads also provide physical separation so that the conductive loop may be deposited or connected using conductive glue with coarse resolution on the RFID tag. In some aspects, where the conductive loop is breakable or when the breakable loop is broken, such as when the thin backing membrane is torn, data in the RFID tag is mechanically reprogrammed.

The microchip incorporated in the medicament source container or the medicament source connector may utilize a passive, inexpensive, disposable, non-contact, non-volatile read/write RFID tag to authenticate a medical supply or component and indicate its use status (such as, used, unused, past expiration or inspection date, number of times used) for single-use and multiple-use medical supplies. The reading/writing zones of a reader/writer are advantageously located to encompass the tags of tagged medical supplies when used in conjunction with an inventory control unit or a capital equipment unit operably coupled to the reader/writer. The RFID reader/writer is attached to or associated with the medicament infusion system and communicates with a CPU of the system. Software resident on the CPU of the system in turn may interface and communicate with other systems like a local area network (LAN), inventory control system, automated charge capture and billing system, medical record system, as well as the Internet and the Web and other Internet- and Web-based applications. Data concerning a smart medical supply or component may also be used by other subsystems of a capital equipment unit, for example, to facilitate and speed a semi-automated pre-use or functional check of the capital equipment unit. A reader/writer could interface to an RFID tag via, among other techniques, inductive coupling (e.g., by using an antenna) or capacitive coupling (e.g., by using conductive carbon ink that picks up electrostatic charges from reader). RFID tags from manufacturers including Texas Instruments, Motorola, Philips, Mitsubishi, Intermec, Micron and SCS may be used.

The amount of data that can be stored in an RFID tag is large enough such that each single tagged item of the medicament infusion system has a unique, individual identification number as well as a batch number. The batch number may form part of the unique identification number or may be separate therefrom. Having enough data storage capability on an RFID tag to assign a unique number to each individual tagged medical supply and component as it makes its way from a manufacturer to a patient enables the creation of powerful databases providing real-time data to improve the efficiency of manufacturing, distribution, warehousing, restocking of medical supplies and components, and reduction of waste. See e.g., U.S. Pat. No. 7,559,483 issued to Hickle et al. on Jul. 14, 2009.

### PROPHETIC EXEMPLARY EMBODIMENTS

### Example 1

### A Medicament Infusion System with Smart Connectors and an Embedded Optical Waveguide Communication System

A medicament infusion system may be a venous infusion system providing a medicament tubing line from a medicament reservoir into a patient catheter. The venous infusion system has a medicament data transmitter that communicates via an optical signaling apparatus and smart connectors, a medicament source connector and a patient recipient connector. The venous infusion system is constructed to verify the patient's identity, the prescribed medication/medicament, and the status of drug delivery. The system is comprised of: a medication source, a medicament data transmitter provided by an optical source, a medication source connector, infusion tubing with an embedded optical fiber in the wall of the tubing, a patient recipient connector with photocell, a cannula and a radio frequency identification (RFID) tag. See FIG. 1. Medication data transmission from the medication source connector via the wall of the infusion tubing is translated by the patient recipient connector and relayed to the RFID tag. A health care worker may use an external (e.g., handheld) radio frequency transceiver to query the RFID tag thus obtaining data to match patient identity with prescribed medication for that patient as well as dosage, rate of flow and the status of drug infusion.

The medicament data transmitter includes an optical signaling apparatus programmed to transmit a signal including medication data through the medication source connector and through the infusion tubing via an embedded waveguide incorporated into the wall of the infusion tubing. For example, medication data may include: the drug name, concentration, total volume, specified flow rate, actual flow rate, date and time flow initiated and the elapsed time of flow. As part of the optical signaling apparatus, a semiconductor optical transmitter, e.g., a light emitting diode made from indium gallium arsenide phosphide (InGaAsP), is directly modulated by varying the electric current to transmit data as optical signals. See e.g., “Fiber-optic communication” available online at http://en.wikipedia.org/wiki/Fiber-optic_communication#Receivers, which is incorporated herein by reference. The semiconductor optical transmitter is attached to the outlet of the medication source reservoir and encoded with corresponding medication data, e.g., the drug name, drug lot number, drug concentration, prescribed dosage and patient name and identification number. See FIG. 1.

The medicament source connector is a smart connector attached to the medication source outlet and aligned with the medicament data transmitter to conduct optical signals via optical fiber incorporated into the wall of the infusion tubing. The medicament source connector includes microcircuitry and a thin-film microbattery to empower the optical transmitter and transmit the medication data. See e.g., U.S. Pat. No. 6,669,683 issued to Santini, Jr. et al. on Dec. 30, 2003 which is incorporated herein by reference. The medicament infusion system transmits the medication data through the wall of the tubing via an embedded waveguide, for example, an optic fiber. Optics fibers for transmission of optical data are available from Corning Inc., Corning N.Y. (see e.g., Corning Fiber Optic Product Info Sheet which is incorporated herein by reference). Methods and materials to manufacture tubing with embedded conductors (e.g., optical fibers) are described (see e.g., U.S. Patent Application No. 2014/0037276 by Carlson published on Feb. 6, 2014 which is incorporated herein by reference). Medication data is received by the patient recipient connector. The patient recipient connector contains microcircuitry and a semiconductor-based photocell, e.g., an indium gallium arsenide photodiode (see e.g., the article, “Fiber-optic communication”, Ibid.) that translates optical signals encoding the medication data and communicates with the patient RFID tag located on or near the cannula of the infusion system. A signal light illuminates on the medicament source connector when communication is established between the medicament data optical transmitter and the patient RFID tag. The patient RFID tag is encoded with patient data including patient identity, medication prescribed, dosage, flow rate, and the date and time of infusion. To verify the correct infusion line and the correct medication are connected to the correct patient a nurse queries the patient RFID tag with a handheld RFID reader. If the medication data from the optical transmitter and the patient data encoded in the RFID tag match a visual validation signal appears on the RFID reader accompanied by a chirp sound. Alternatively if the medication data and the patient data do not correspond an error signal, e.g., red light, displays on the RFID reader. If, for example, the medication data received from the medication source is incompatible with the RFID patient data, the patient recipient connector will stop the flow of medication from the medication source. Control circuitry in the patient recipient connector may prevent medication flow by closing an electronic valve in the patient recipient connector.

### Example 2

### A Medicament Infusion System with Smart Connectors and Embedded Circuitry to Prevent Errors in Medication Delivery

A medicament infusion system is constructed with an integrated communication system which transmits medicament/medication information from the medication source reservoir to the patient injection site via the infusion tubing. Data on the prescribed infusion medication in the source reservoir is compared to data on the patient receiving the medication. The patient data is encoded in a radio frequency identification (RFID) tag. The combined medication source data and patient recipient data and comparisons of the data may be obtained by healthcare workers using a handheld device or using a bedside device to query of the RFID tag.

The medicament infusion system includes a medication source, for example, a medication fluid in an infusion bag with an outlet at the bottom with fittings for connection of infusion tubing, bubble traps and flow regulators. See e.g., U.S. Pat. No. 8,075,514 issued to Butterfield et al. on Dec. 13, 2011 and U.S. Patent Appl. No. 2014/0107579 by Lanigan et al. published on Apr. 17, 2014 which is incorporated herein by reference. A microchip is incorporated in the medication infusion bag which encodes medication information, for example, the prescription information, patient identity, dose or concentration, schedule of administration, date and time. Microchip data carriers may be used for identification and information about drug sources. See e.g., U.S. Pat. No. 7,559,483 issued to Hickle et al. on Jul. 14, 2009. A medicament source smart connector links the medication fluid outlet and the microchip (via electronic connectors) to the infusion tubing. The smart connector (medication source connector) contains microcircuitry and a thin film microbattery to empower the infusion bag microchip and transmit medication information via embedded conductors within the wall of the infusion tubing to a second, patient-associated smart connector. See e.g., U.S. Patent Application No. 2014/0037276 by Carlson published on Feb. 6, 2014 which is incorporated herein by reference. A patient recipient smart connector receives electronic data regarding the medicament source from the medicament data transmitter and medicament source connector through embedded conductors within the infusion tubing wall and translates the data to a RFID tag proximal to the infusion line cannula. See FIG. 1. The patient recipient smart connector includes a radio frequency transponder for communicating with the RFID tag. See e.g., U.S. Pat. No. 8,075,514, Ibid. The patient RFID tag is attached to the cannula or nearby the patient. The patient RFID tag is encoded with patient information. For example, patient admission information such as patient name, identification, physician, birthdate, day and time of admission may be entered onto the RFID tag at the time of admission. Data regarding the medicament infusion apparatus, e.g., venous cannula, and the infusion day and time may also be encoded on the RFID tag. Microprocessors in the patient recipient connector signal when a communication link is established between the patient RFID and the medication source data transmitter in the medication source connector. Moreover, the smart connector microprocessors compare medication information from the medicament source data transmitter with the patient information on the RFID tag. Comparisons include matching patient identity with medication prescription and medication source. The status of the medicament infusion system may be checked by a healthcare worker using an external RFID transponder to query the patient RFID tag. The external RFID transponder will indicate a positive or negative signal depending upon a successful connection of the medicament tubing line and agreement between the medicament source data and the patient recipient data. A positive signal will electronically open flow valves and a negative signal will electronically close flow valves to control medicament flow between the medicament source and the patient recipient.

The medicament infusion system may also be queried at later time points to monitor the status of the medicament infusion. For example, the healthcare worker may determine what volume of medicament solution has been infused, what has been the rate of infusion, and what the total amount of medicament has been delivered intravenously.

### Example 3

### A Medicament Infusion System for Initiating Infusion of Potassium Chloride in a Patient

A patient with low levels of potassium in blood is treated with intravenous potassium chloride using a medicament infusion system with smart connectors including a medicament source connector and a patient recipient connector to prevent errors in administration of intravenous drugs. A venous infusion system with a medicament data transmitter and an optical signaling apparatus and smart connectors verifies the patient's identity, the prescribed medication, and the status of drug delivery with the data received from the medicament data transmitter at the medicament source. A patient with less than 2 mEq/L of potassium in their serum is prescribed an intravenous infusion of potassium chloride. The emergency medicine physician recommends potassium chloride (KCl) be infused at a rate of 40 mEq/hour with the total dose not to exceed 400 mEq in 24 hours. KCl in 5% dextrose for injection (40 mEq/liter) in 1000 mL is prescribed.

The hospital pharmacist enters the patient's name and identification, the prescribed medication, and detailed dosage and schedule information into a computer which encodes the information on a microchip which acts as a medication data transmitter attached to the infusion bag containing the prescribed KCl solution. The patient has been admitted to the hospital and patient's identification, date and time of admission are entered on a computer and encoded in a RFID tag which is attached to the patient near the cannula for the infusion system or is attached on the patient connector of the infusion system. The medicament infusion system is set up by a healthcare worker who attaches infusion tubing with smart medicament source connector and patient recipient connector at each end to the KCl infusion bag. The medicament source smart connector links with the outlet for the medicament infusion bag and connects electronically with the medicament data transmitter microchip on the infusion bag. The medicament source smart connector includes a microbattery that empowers the medicament data transmitter microchip to transmit electronic signals encoding the medication and prescription data detailed above from the medicament data transmitter microchip to the medicament source connector on the infusion tubing. An electrical conductor embedded in the wall of the infusion tubing is connected through the medicament source smart connector to the patient recipient smart connector. The infusion cannula in the patient with an attached patient RFID tag is connected to the patient recipient smart connector of the infusion tubing. Signal transmission from the medication source microchip to the medicament source smart connector and the patient recipient smart connector via the electrical connection in the wall of the infusion tubing may be completed to the RFID tag. Successful signal transmission is indicated by a signal light on the medication source smart connector. Before inserting the cannula in the patient the healthcare worker queries the RFID tag with an RFID transponder. If the patient information and the medication information match a “go” indication is given. With a “go” signal the healthcare worker inserts the cannula in the patient's vein and initiates the infusion. Alternatively if a “stop” signal is indicated by the system, the medication data and the patient information data must be verified prior to introducing the cannula and initiating infusion.

The infusion system may also be queried at later time points to monitor the status of the infusion. For example, the healthcare worker may determine what volume KCl solution has been infused, what has been the rate of infusion, and what the total amount of KCl has been delivered intravenously.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if a surgeon determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those having ordinary skill in the art will recognize that optical aspects of implementations will typically employ optically-oriented hardware, software, and or firmware.

In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microdigital processing unit configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A medicament infusion system comprising:
a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end;
a medicament data transmitter operably connected to communicate with the medicament source connector, the medicament data transmitter configured to transmit medicament source data to the medicament source connector;
a communication connection embedded within the wall of the cylindrical tubing member, the communication connection configured to transmit medicament source data from the medicament source connector to the patient recipient connector;
a wireless tag operably connected to communicate with the patient recipient connector, the wireless tag configured to transmit the medicament source data; and
a controller operably connected to receive medicament source data from the wireless tag, the controller including circuitry to determine a connection status between the medicament source connector and the patient recipient connector.

2. The system of claim 1, wherein the controller includes circuitry to receive patient recipient data to determine the connection status between the medicament source connector and the patient recipient connector.

3. The system of claim 1, comprising:
a signaling system operably connected to the controller and configured to transmit a wireless medicament delivery signal to the wireless tag, the medicament delivery signal comprising delivery instructional data for the source medicament based on the determined connection status between the medicament source connector and the patient recipient connector.

4. The system of claim 3, comprising:
one or more valves operably connected to the cylindrical tubing member, the one or more valves configured to modify flow of a medicament through the cylindrical tubing member between the medicament source reservoir and the patient recipient in response to the delivery instructional data.

5. The system of claim 3, wherein the communication connection is configured to transmit the delivery instructional data from the patient recipient connector to the medicament source connector.

6.-10. (canceled)

11. The system of claim 1, comprising:
an alert signaling system in communication with the controller to notify a caregiver of the connection status between the medicament source connector and the patient recipient connector, wherein the controller receives and compares the patient recipient data and the medicament source data from the wireless tag to determine the connection status between the medicament source connector and the patient recipient connector.

12.-13. (canceled)

14. The system of claim 1, wherein the medicament data transmitter is in contact with the medicament source reservoir.

15. The system of claim 1, wherein the medicament data transmitter is communicatively linked to the medicament source reservoir.

16. The system of claim 2, wherein the wireless tag includes the patient recipient data, and is configured to transmit the patient recipient data to the controller.

17.-18. (canceled)

19. The system of claim 1, wherein the controller is included within a handheld electronic device.

20. The system of claim 1, wherein the controller is included within an electronic device proximal to the patient recipient.

21. The system of claim 1, wherein the wireless tag is configured to transmit at least a portion of the medicament source data in response to reception of an RF signal by the wireless tag.

22. The system of claim 21, wherein the RF signal comprises a request for a first portion of the medicament source data, and wherein the wireless tag is configured to transmit at least a second portion of the medicament source data corresponding to at least a part of the first portion of medicament source data.

23. The system of claim 21, wherein the medicament data transmitter is configured to transmit the second portion of the medicament source data in response to reception of a wireless signal by the wireless tag.

24. The system of claim 1, comprising one or more intermediate cylindrical tubing members having a second communication connection embedded within a second wall defining a second interior passage, the second cylindrical tubing member having one or more intermediate connectors configured to connect the one or more intermediate cylindrical tubing members between the medicament source connector and the patient recipient connector, wherein the one or more intermediate connectors are configured to transmit the medicament source data between the intermediate cylindrical tubing members, the medicament source data configured to be transmitted between the medicament source connector and the patient recipient connector via the second communication connection and the one or more intermediate connectors.

25. The system of claim 24, wherein the medicament source data is communicated to the wireless tag associated with the patient recipient.

26. The system of claim 1, comprising two or more intermediate cylindrical tubing members fluidically and communicatively coupled to the medicament source connector and the patient recipient connector on the cylindrical tubing member by two or more intermediate connectors.

27. The system of claim 26, wherein the two or more intermediate connectors are configured to transmit the source medicament data through a second wall of the intermediate cylindrical tubing members.

28. The system of claim 1, wherein the medicament data transmitter includes an electrical transmitter or an optical transmitter configured to transmit the medicament source data electrically or optically, to the medicament source connector.

29. The system of claim 1, wherein the medicament data transmitter includes a wireless transmitter configured to transmit the medicament source data wirelessly by near field propagation to the medicament source connector.

30.-31. (canceled)

32. The system of claim 1, wherein the communication connection comprises electrical conductors embedded within the wall of the tubing member.

33. The system of claim 1, wherein the communication connection comprises an optical waveguide embedded within the wall of the tubing member.

34. The system of claim 33, wherein the optical waveguide comprises at least a portion of the wall of the tubing member.

35. The system of claim 33, wherein the optical waveguide comprises at least a portion of the interior passage of the tubing member.

36. The system of claim 33, comprising an optical source to transmit the data through the optical waveguide.

37.-38. (canceled)

39. A method comprising:
transmitting medicament source data along a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end;
transmitting the medicament source data from a medicament data transmitter to the medicament source connector, wherein the medicament data transmitter is operably connected to communicate with the medicament source connector;
transmitting the medicament source data on a communication connection embedded within the wall of the cylindrical tubing member, the communication connection operably connected to communicate between the medicament source connector and the patient recipient connector;
transmitting the medicament source data from the patient recipient connector operably connected to communicate to a wireless tag associated with the patient recipient;
receiving at a controller the medicament source data from the wireless tag operably connected to communicate to the controller; and
determining at the controller a connection status between the medicament source connector and the patient recipient connector.

40. The method of claim 39, comprising:
transmitting patient recipient data to the controller; and
determining at the controller the connection status between the medicament source connector and the patient recipient connector.

41. The method of claim 39, comprising:
transmitting from a signaling system operably connected to the controller, a wireless medicament delivery signal transmitted to the wireless tag, the wireless medicament delivery signal comprising delivery instructional data for the source medicament based on the determined connection status between the medicament source connector and the patient recipient connector.

42. The method of claim 41, comprising:
modifying flow of a medicament between the medicament source reservoir and the patient recipient with one or more valves in response to the delivery instructional data.

43. The method of claim 41, transmitting via the communication connection the delivery instructional data from the patient recipient connector to the medicament source connector.

44. The method of claim 40, wherein the patient recipient data comprises at least one of a patient identifier, a patient treatment parameter, an identifier of a third party payer associated with the patient, and an identifier of a medical personnel associated with the patient.

45. The method of claim 44, wherein the patient treatment parameter comprises at least one of an identifier of a source medicament prescribed for the patient, an indication that the source medicament is authorized for delivery to the patient by the medicament infusion system, a prescribed delivery time for the source medicament, a prescribed delivery dosage for the source medicament, a prescribed delivery rate for the source medicament, and a prescribed composition of the source medicament.

46. The method of claim 39, comprising:
receiving and comparing the patient recipient data and the medicament source data from the wireless tag at the controller to determine the connection status between the medicament source connector and the patient recipient connector; and
notifying a caregiver of the connection status between the medicament source connector and the patient recipient connector by a signaling system.

47. The method of claim 39, comprising:
providing one or more intermediate cylindrical tubing members having a second communication connection embedded within a second wall defining a second interior passage, the second cylindrical tubing member having one or more intermediate connectors configured to connect the one or more intermediate cylindrical tubing members between the medicament source connector and the patient recipient connector, wherein the one or more intermediate connectors are configured to transmit the medicament source data between the intermediate cylindrical tubing members, the medicament source data configured to be transmitted between the medicament source connector and the patient recipient connector via the second communication connection and the one or more intermediate connectors.

48. The method of claim 47, comprising communicating the medicament source data to the wireless tag associated with the patient recipient.

49. The method of claim 39, comprising fluidically and communicatively coupling through two or more walls of two or more intermediate cylindrical tubing members by two or more intermediate connectors to the medicament source connector and the patient recipient connector on the cylindrical tubing member.

50. The method of claim 39, comprising communicatively linking the source reservoir to the medicament data transmitter wherein the source reservoir comprises the medicament data transmitter.

51. The method of claim 39, comprising transmitting via the medicament data transmitter at least a portion of the medicament source data in response to reception of a wireless signal from the wireless tag.

52. The method of claim 39, comprising:
transmitting the data through the medicament data transmitter electrically or optically to the medicament source connector.

53. The method of claim 39, comprising:
transmitting the data through the medicament data transmitter wirelessly by near field propagation to the medicament source connector.

54. The method of claim 39, comprising: receiving data electrically, optically, or wirelessly by the RFID tag at the patient recipient connector.

55. (canceled)

56. The method of claim 39, comprising:
embedding electrical conductors on the communication connection within the wall of the tubing member.

57. The method of claim 39, comprising:
embedding an optical waveguide on the communication connection within the wall of the tubing member.

58. The method of claim 57, comprising:
transmitting light through the optical waveguide to transmit data through at least a portion of a wall of the cylindrical tubing member.

59. The method of claim 57, comprising: transmitting light through the optical waveguide to transmit data through at least a portion of the interior passage of the cylindrical tubing member.

60. The method of claim 39, comprising:
locating the wireless tag on or near a patient and adjacent to the patient recipient connector of the tubing member.

61. The method of claim 39, comprising:
locating the wireless tag on a cannula at least a portion of which is configured to be inserted within the patient recipient.

62.-64. (canceled)

65. A medicament infusion system comprising:
a cylindrical tubing member having a wall defining an interior passage, a medicament source end, and a patient recipient end, the tubing member having a medicament source connector configured to connect to a medicament source reservoir at the medicament source end and a patient recipient connector configured to connect to a patient recipient at the patient recipient end;
a medicament data transmitter operably connected to communicate with the medicament source connector, the medicament data transmitter configured to transmit medicament source data to the medicament source connector;
an optical waveguide embedded within the wall of the cylindrical tubing member, the optical waveguide configured to transmit medicament source data from the medicament source connector to the patient recipient connector;
a wireless tag operably connected to communicate with the patient recipient connector, the wireless tag configured to transmit the medicament source data; and
a controller operably connected to receive medicament source data from the wireless tag, the controller including circuitry to determine a connection status between the medicament source connector and the patient recipient connector.

66. (canceled)
